(19) United States
(12) Patent Application Publication
(10) Pub. No.: US 2005/0171478 A1
(43) Pub. Date: Aug. 4, 2005

(54) Catheter system for crossing total occlusions in vasculature

(76) Inventors: Matthew R. Selmon, Redwood City, CA (US); Kurt Sparks, Redwood City, CA (US); Ray Betelia, Redwood City, CA (US); Ben Clark, Redwood City, CA (US); Jason Kaiser, Redwood City, CA (US); Rob Deckman, Redwood City, CA (US); Erik Thai, Redwood City, CA (US)

Correspondence Address: Shemwell Gregory & Courtney LLP, Suite 201, 4880 Stevens Creek Boulevard, San Jose, CA, 95129, US

(21) Appl. No.: 10/823,416

(22) Filed: Apr. 12, 2004

Related U.S. Application Data
(63) Continuation-in-part of application No. 10/308,568, filed on Dec. 3, 2002, now Pat. No. 6,719,725.
Application No. 10/308,568 is a continuation of application No. 09/765,777, filed on Jan. 19, 2001, now Pat. No. 6,511,458.
Application No. 09/765,777 is a continuation of application No. 09/440,308, filed on Nov. 17, 1999, now Pat. No. 6,235,000.
Application No. 09/440,308 is a division of application No. 09/006,563, filed on Jan. 13, 1998, now Pat. No. 6,231,546.

(51) Int. Cl. A61M 5/178

(52) U.S. Cl. 604/164.01

(57) ABSTRACT

Medical devices/methods are described that include catheter systems for crossing total occlusions in vasculature. The catheter systems are for use with numerous guide wires to direct the wires from the extraluminal or subintimal space back into a blood vessel lumen after the wire has entered the subintimal space. The catheter systems comprise a catheter body having at least one lumen configured to track over a wire. An endpiece that includes at least one port in communication with the lumen couples to the distal region of the catheter body. A working element is configured to deploy through the port for delivery from a location within the extraluminal space to locations within the vessel true lumen when advanced distally through the port. A distal region of the catheter body assumes first or second configurations when the working element is retracted into the catheter body.

### RELATED APPLICATIONS

This application is a continuation-in-part application of U.S. patent application Ser. No. 10/308,568, filed Dec. 3, 2002, which is a continuation of U.S. patent application Ser. No. 09/765,777, filed Jan. 19, 2001, now U.S. Pat. No. 6,511,458, which is a continuation of U.S. patent application Ser. No. 09/440,308, filed Nov. 17, 1999, now U.S. Pat. No. 6,235,000, and which is a division of U.S. patent application Ser. No. 09/006,563, filed Jan. 13, 1998, now U.S. Pat. No. 6,231,546.

### TECHNICAL FIELD

The disclosure herein relates generally to medical devices and methods. In particular, this disclosure relates to systems, methods and procedures for crossing chronic total occlusions in vasculature.

### BACKGROUND

Cardiovascular disease is a leading cause of mortality worldwide. Cardiovascular disease can take many forms, and a variety of specific interventional and pharmaceutical treatments have been devised over the years with varying levels of success.

A particularly troublesome form of cardiovascular disease results when a blood vessel becomes totally occluded with atheroma or plaque, referred to as a chronic total occlusion (CTO). Until recently chronic total occlusions have typically been treated by performing a bypass procedure where an autologous or synthetic blood vessel is anastomotically attached to locations on the blood vessel upstream and downstream of the occlusion. While highly effective, such bypass procedures are quite traumatic to the patient.

Recently, catheter-based intravascular procedures have been utilized to treat chronic total occlusions with increasing success. Catheter-based intravascular procedures include angioplasty, atherectomy, stenting, and the like, and are often preferred because they are much less traumatic to the patient. Before such catheter-based treatments can be performed, however, it is usually necessary to cross the occlusion with a guide wire to provide access for the interventional catheter.

In some instances, crossing the occlusion with a guide wire can be accomplished simply by pushing the guide wire through the occlusion. After being advanced through the occlusion, the guide wire emerges in the blood vessel lumen and provides the desired access path. In many cases, however, the guide wire inadvertently penetrates into the subintimal space between the intimal layer and the adventitial layer of the blood vessel as it attempts to cross the occlusion. Once in the subintimal space, it is very difficult and in many cases impossible for a physician/user to direct the guide wire back into the blood vessel lumen. In such cases, it will usually be impossible to perform the catheter-based intervention and other, more traumatic, procedures may have to be employed. Catheters for use in treating chronic total occlusions are described in U.S. Pat. Nos. 4,405,314, 4,947,864, 5,183,470, 5,190,528, 5,287,861, 5,409,019, 5,413,581, 5,429,144, 5,443,497, and 5,464,395 as well as in International Publication Numbers WO 97/13463 and WO 97/13471. For these reasons, there is a need to provide apparatus and methods that facilitate the crossing of chronic total occlusions with guide wires.

### BRIEF DESCRIPTION OF THE DRAWINGS

FIG. 1 shows a section of artery and the tissue layers that form the artery.

FIG. 2A is a section of a diseased artery that shows detail of the normal tissue of the arterial wall along with a total occlusion.

FIG. 2B is a section of a diseased artery that shows an arterial wall with a modified tissue structure that can result from the presence of the total occlusion.

FIG. 3A, 3B, 3C, 3D, and 3E shows a procedure for crossing a total occlusion using a catheter system, under an embodiment.

FIG. 4 is a distal region of a deflecting catheter, under an embodiment.

FIG. 5 is a distal region of a deflecting catheter, under an alternative embodiment.

FIG. 6 shows a distal region of a deflecting catheter, under another alternative embodiment.

FIG. 6A shows a distal region of a deflecting catheter that assumes a straight configuration in the retracted state, under an alternative embodiment.

FIG. 6B shows a distal region of a deflecting catheter that assumes a curved configuration in the retracted state, under an alternative embodiment.

FIG. 7 is a deflecting catheter system, under an embodiment.

FIG. 8 is a cross-sectional view of a distal region of the defecting catheter system that includes a cannula in a retracted configuration, under an embodiment.

FIG. 9 is a cross-sectional view of a distal region of the defecting catheter system that includes a cannula in an advanced configuration, under an embodiment.

FIG. 10 is a cross-sectional view of a proximal region of the defecting catheter system that includes a proximal hub, under an embodiment.

FIG. 10A and FIG. 10B are cross-sectional views of a proximal actuation handle with a locking mechanism that prevents inadvertent deployment of the cannula, under an embodiment.

FIG. 11A and FIG. 11B show rotational keying in cross-sectional views of a proximal region of the defecting catheter system, under an embodiment.

FIG. 12 shows rotational keying in cross-sectional views of a distal region of the defecting catheter system, under an embodiment.

FIG. 13 is a catheter system that includes a catheter shaft having a distal end port and a proximal phased array ultrasound device, under an embodiment.

FIG. 14A shows an ultrasound visualization system deployed beyond a distal end of the catheter shaft to image surrounding tissue, under an embodiment.

FIG. 14B shows an ultrasound visualization system deployed to image surrounding tissue from a window within the nosecone of the catheter system, under an embodiment.

FIG. 15 shows an ultrasound visualization system deployed within a cannula of the catheter shaft to image surrounding tissue, under an embodiment.

FIG. 16 shows a composite distal end termination of a braided catheter shaft that includes a nosecone, under an embodiment.

### DETAILED DESCRIPTION

Devices and methods are provided below that include catheters, guides, and/or other apparatus for use in crossing total occlusions in vasculature. The total occlusions are also referred to as total vascular occlusions or chronic total occlusions (CTOs). The devices/methods include medical devices for use by physicians/users with conventional and/or specialized guide wires to direct or redirect the guide wire from the subintimal space back into the blood vessel lumen after the guide wire has entered the subintimal space. These devices/methods are useful in the treatment of coronary artery disease in coronary arteries as well as other blood vessels and should be capable of being performed with or without imaging. The devices/methods are also useful for applications in other arteries and veins, such as the treatment of peripheral vascular diseases.

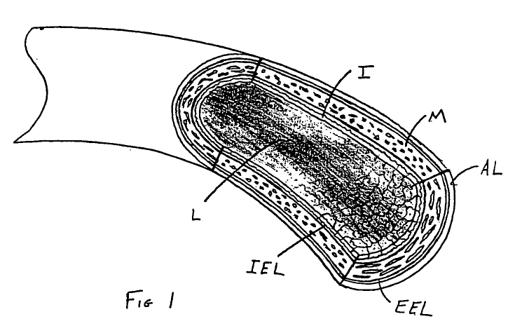

FIG. 1 shows a section of artery A and the tissue layers that form the artery A. A normal (non-diseased) artery A comprises an arterial wall having a number of layers. The innermost layer is referred to herein as the intimal layer I which includes the endothelium, the subendothelial layer, and the internal elastic lamina IEL. A medial layer M is concentrically outward from the internal elastic lamina IEL, an external elastic lamina layer EEL is concentrically outward from the medial layer M, and an adventitial layer AL is the outermost layer. Beyond the adventitial layer AL is extravascular tissue. As used hereinafter, the region between the intimal layer I and the adventitial layer AL, generally including the medial layer M, is referred to as the subintimal space, but the subintimal space can include additional tissue types/layers as described below. This definition of subintimal space used herein is in addition to any meaning(s) provided by those skilled in the art.

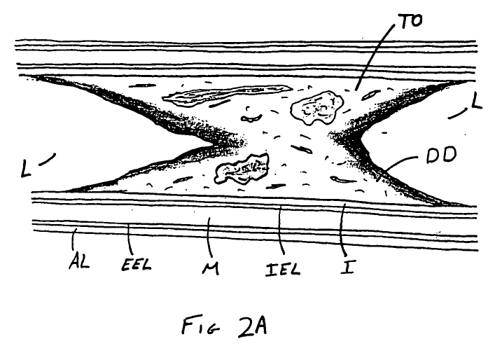

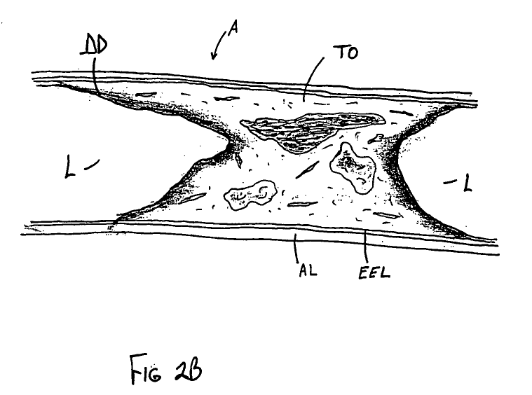

FIG. 2A is a section of a diseased artery A that shows detail of the normal tissue of the arterial wall along with a total occlusion TO. FIG. 2B is a section of a diseased artery A that shows an arterial wall with a modified tissue structure that can result from the presence of the total occlusion TO. With reference to FIG. 2B, a diseased artery A with a total occlusion TO comprises an arterial wall having a modified structure as compared to the tissue layers of a normal artery. The innermost layer is referred to as diffuse disease DD. The diffuse disease DD layer may range in thickness from approximately 50 microns to 500 microns, but is not limited to this thickness in heavily diseased arteries. A medial layer M may be located concentrically outward from the total occlusion TO and diffuse disease DD. In heavily diseased vessels the medial layer M may have eroded and may not be evident, as shown in FIG. 2B. An external elastic lamina EEL may be located concentrically outward from either the medial layer M or total occlusion TO or diffuse disease DD, and an adventitial layer AL is the outermost layer. As used hereinafter, the region within the diffuse disease DD and bounded by the adventitial layer AL is also referred to as the subintimal space, where this additional definition of subintimal space is in addition to any meaning(s) provided by those skilled in the art. The subintimal space is the region through which the wires, deflecting catheters, and other catheters described herein pass when crossing a total occlusion.

A total occlusion TO may comprise atheroma, plaque, thrombus, and/or other occluding materials normally associated with cardiovascular disease. By “total” occlusion, it is meant that the occluding material occludes substantially the entire lumen L of the artery or other blood vessel so that blood flow through the vessel is substantially blocked or stopped. The catheter systems and methods described herein are generally used with patients in which the totally occluded artery is not immediately life threatening since the tissue distal to the occlusion will often receive oxygenated blood from collateral arteries. Usually, however, the blood supply in regions distal to the occlusion will be insufficient and it will be desirable to treat the occlusion by an intravascular intervention, such as angioplasty, atherectomy, stenting, or the like, to restore blood flow through the affected vessel.

Total occlusions are crossed by positioning a guide wire, or blunt dissection catheter (as described in U.S. Pat. Nos. 5,968,064, 6,217,549, 6,398,798, 6,508,825, 6,599,304, and 6,638,247, for example) at the proximal end of the occlusion and advancing the device through the occlusion using conventional interventional methods. Crossing of the total occlusions is defined herein, in addition to any meanings provided by those skilled in the art, as establishing a longitudinal path from the proximal end of the occlusion to the distal end of the occlusion. The longitudinal path of the guide wire and/or blunt dissection catheter is to remain as central as possible within the occluded vessel and emerge in the true lumen of the vessel after having traversed the occlusion. In practice however, the guide wire or blunt dissection catheter often tracks an eccentric pathway through the occlusion and, after having been advanced beyond the distal end of the occlusion itself, is contained within a layer of vascular tissue that is distal to the terminal end of the occlusion. A dissection track of this type is generally referred to as a subintimal track, i.e. between the intimal layer and adventitial layer of the vessel, and is typically contained within the medial layer but is not so limited (FIGS. 1, 2A, and 2B).

However, more often the disease state that forms a total occlusion erodes the inner layers of the vessel wall at the site of the occlusion, as described above with reference to FIG. 2B. Further, the erosion of the vessel wall and the disease state often does not abruptly end at either of the proximal or distal ends of the occlusion; instead, the disease state also lines the vessel wall tapering up to and away from the occlusion. Accordingly, the structure of the vessel section that is proximal and distal to the terminal ends of the occlusion is often diffusely diseased. In these diseased vessel sections proximal and distal to the total occlusion, the intimal layer (I), internal elastic lamina layer (IEL) and the medial layer (M) may not be present, and are often replaced with a layer of diffuse disease (DD) that comprises at least one of atheroma, plaque, thrombus, fatty and fibo-calcific deposits/tissue. All of this diseased tissue is typically contained within the external elastic lamina (EEL) and the outer-most boundary of the vessel, the adventitial layer (AL), but is not so limited.

Therefore, taking into consideration that the vessel segment in the region of the occlusion may be either of normal structure (FIG. 2A), or of diseased structure (FIG. 2B), the subintimal track which is distal to the occlusion may be described in two ways: within a normal vessel, the subintimal track is described as being bounded by the adventitial layer (AL) and the intimal layer (I), i.e. within the vessel wall, and usually thought to be within the medial layer (M); and, within a diseased vessel, the subintimal track is described as being within a layer of diffuse disease (DD) that is also externally bounded by the external elastic lamina (EEL) or adventitial layer (AL).

Note, also, that there is a difference between a dissection tract that is contained within the total occlusion, and a dissection tract that is propagated beyond the total occlusion. When the dissection tract is propagated beyond the total occlusion, both types of subintmal tracks described above will be defined as being extra-luminal, i.e. outside the bounds of the vessel true lumen that is distal to the occlusion. The term “extra-luminal” only has relevance when the dissection track has been advanced beyond the distal end of the total occlusion. Note that a dissection track that is contained within a total occlusion can have no reference to an extra-luminal location, since no physical lumen exists within the occlusion. Hence for further reference, the subintimal tract distal to the occlusion is defined as extra-luminal, in addition to any meaning(s) provided by those skilled in the art.

Also notable is the difference between intra-vascular and extra-vascular locations. Since all dissection tracts described herein are contained within the boundary of the blood vessel, e.g. within the adventitial layer (AL), these are considered “intra-vascular”. Accordingly, all locations outside of the adventitial layer (AL) are termed “extra-vasculai”. Extra-vascular locations are not material to the discussions described herein.

Having now established the different structures found in diseased vessels, from the subintimal, extra-luminal locations previously described, a passage or pathway is formed from these subintimal locations to the true lumen of the vessel via methods described herein. In the methods of an embodiment, a guide wire is deflected using a deflecting catheter. Typically, the deflecting catheter is advanced over a proximal end of the guide wire and advanced into the track within the subintimal space. The guide wire and the deflecting catheter are then manipulated so that the guide wire is deflected laterally through the intimal layer or diffuse disease back into the blood vessel lumen at a point distal to the occlusion. Such deflecting catheters also support the guide wire as it is advanced into and/or through the track, i.e. the catheter can enhance the pushability of the guide wire when it is advanced forward through any resisting material.

Alternatively, the guide wire which is initially positioned within the track in the subintimal space may be withdrawn through the deflecting catheter and exchanged for a second wire or other device suitable for penetrating through the intimal layer or diffuse disease back into the blood vessel lumen. The guide wires and/or deflecting catheters and other catheters can be freely exchanged over or through one another in a conventional matter without departing from the methods described herein.

In an embodiment, the physician/user determines when the guide wire and/or deflecting catheter is positioned distal to the total occlusion so that the guide wire can be returned to the blood vessel lumen beyond or distal to any occlusions. Most simply, such position determination can be made by fluoroscopically imaging the blood vessel in a conventional matter.

Alternatively or additionally to such fluoroscopic imaging, intravascular imaging, e.g. intravascular ultrasonic imaging (IVUS), and a variety of optical imaging modalities, such as optical coherence tomography (OCT), are used. For example, an ultrasonic imaging guide wire may be used to initially access the subintimal space and/or may be exchanged for the guide wire which is used to access the subintimal space. Alternatively, the imaging guide wire may be advanced within a lumen of the catheter, or within a cannula to a distal location of the catheter suitable for viewing the surrounding vascular tissue. An imaging guide wire present in the subintimal space may readily detect the presence or absence of occluding material within the blood vessel lumen. When the transition from occluding material to normal arterial tissue is detected, it is known that the position of the guide wire has advanced beyond that of a distal region of the total occlusion.

Alternatively, an imaging system or select imaging components like those described in U.S. Pat. Nos. 5,000,185 and 4,951,677 can be carried on and/or within the catheter system and/or advanced within a lumen of the deflecting catheter to a distal position within the catheter, wherein the surrounding tissue is imaged to determine if the catheter has been advanced beyond a distal region of the total occlusion. The catheter system of an alternative embodiment may house and translate the imaging system or components within the lumen of the cannula itself so that both the cannula and imaging system are independently advanced distally and retracted proximally.

After a passage is formed back from the sub-intimal track into the blood vessel lumen and a wire is in place across the total occlusion, the wire is available for use as a guide wire in positioning interventional and diagnostic catheters across the total occlusion. Most comrnmonly, interventional catheters are positioned across the total occlusion for treating the occlusion. Interventional catheters include, for example, angioplasty balloon catheters, rotational atherectomy catheters, directional atherectomy catheters, and stent-placement catheters, but are not so limited.

Wire deflecting in the catheter system of an embodiment comprises deflecting a cannula from the subintimal space back into the blood vessel lumen and thereafter passing the wire through a path defined/formed by the cannula, typically via a lumen within the cannula. The cannula is advanced over the wire after the wire is disposed within the subintimal space. Deflecting of the cannula in an embodiment comprises advancing a resilient (preformed) curved end of the cannula from a constraining lumen of the catheter into the blood vessel lumen, as described below.

Wire deflecting in alternative embodiments of the catheter system comprises advancing a deflecting catheter over a wire that was previously advanced into the subintimal space. The cannula is subsequently advanced through a lateral opening of the deflecting catheter and penetrated through the intimal layer or diffuse disease to define a path for the guide wire back into the blood vessel lumen.

Wire deflecting in other alternative embodiments comprises advancing a deflecting catheter over a wire which was previously advanced into the subintimal space. The cannula is subsequently advanced through a distal opening of the deflecting catheter and penetrated through the intimal layer or diffuse disease to define a path for the guide wire back into the blood vessel lumen. Steerable and other actively deployed cannulas may also be used.

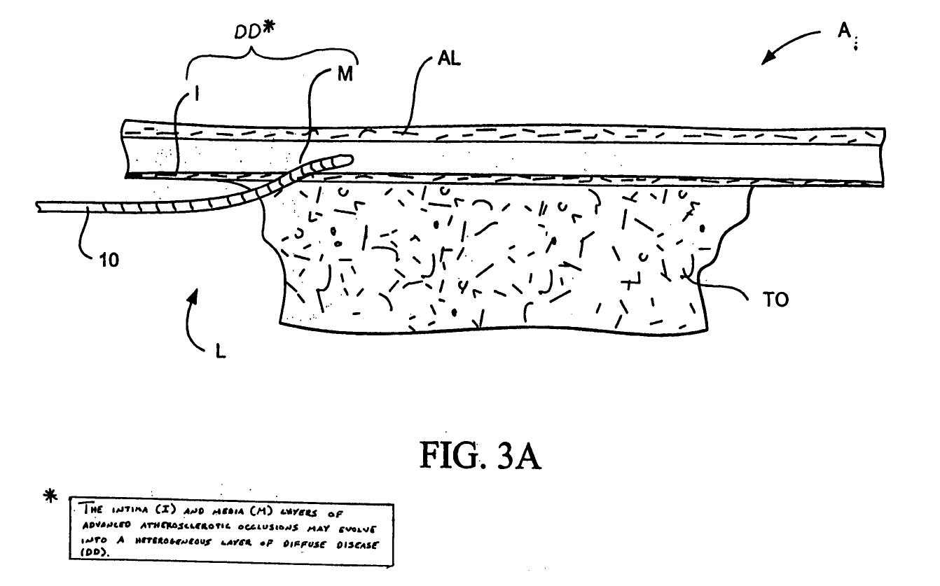

FIG. 3A, 3B, 3C, 3D, and 3E shows a procedure for crossing a total occlusion using a catheter system, under an embodiment. The catheter system includes a deflecting catheter 20 and at least one wire 10, or guide wire 10, but is not so limited. With reference to FIG. 2A and FIG. 2B, this procedure is performed in an upper portion of the artery, but is not so limited. Referring to FIG. 3A, a wire 10 is advanced through the lumen L of the artery A until it encounters material of a total occlusion TO, as described above. At that time, it is possible that the wire 10 will advance through the occlusion TO without deflecting into the blood vessel wall. Should that occur, subsequent repositioning of the guide wire according to the methods of the present invention may not be necessary.

More usually, however, the wire 10 will advance into the subintimal space within either the medial layer M, or the diffuse disease DD, as shown in FIG. 2B (as described above, the intimal and medial layers of advanced atherosclerotic occlusions may evolve into a heterogeneous layer of diffuse disease DD). The intimal layer I and adventitial layer AL together define a tissue plane through which the wire 10 can naturally pass as the wire 10 is pushed distally from its proximal end. Alternatively, the wire 10 may be advanced through the diffuse disease DD and take a similar pathway through the occlusion TO. The wire 10 will continue to advance until the distal tip of the wire 10 passes beyond the distal end of the total occlusion TO, as shown in FIG. 3B. The distal tip of wire 10 can axially advance well beyond the total occlusion until advancement is ceased by the physician/user.

FIG. 3B shows the guide wire 10 advancing without support. In some instances, however, the guide wire 10 may encounter significant resistance as it enters and/or passes through the space between the intimal layer I and the adventitial later AL, or the diffuse disease DD. If resistance is encountered, the deflection catheter 20 may be used to support and enhance the pushability of the guide wire 10 by advancing the deflection catheter 20 to a location just proximal of the distal tip of the guide wire 10, as shown in FIG. 3C. The guide wire 10 and catheter 20 may then be advanced sequentially, e.g. advancing the guide wire 10 a short distance followed by advancing the catheter 20 to a location just proximal of the distal tip of the guide wire 10, and so on.

Regardless of the procedure used, however, once the guide wire 10 is advanced to a point that positions the distal tip beyond the total occlusion TO, deflecting catheter 20 is advanced over the wire 10, by coaxial introduction over the proximal end of the wire 10, until it approaches the total occlusion TO, as shown in FIG. 3B. The deflecting catheter 20 is then further advanced over the wire 10 until its distal tip also extends beyond the total occlusion TO, as shown in FIG. 3D. The deflecting catheter 20 of an embodiment includes at least one mechanism for laterally deflecting the guide wire 10 so that the guide wire 10 can pass in a radially inward direction through the intimal layer I or the diffuse disease DD back into the blood vessel lumen L.

The deflection mechanism of an embodiment takes a variety of forms as described below. For example, referring to FIG. 3D, the deflection mechanism of an embodiment includes a lateral port 22 in the deflecting catheter 20. The guide wire 10 can be retracted so that its distal tip lies proximal to the lateral port 22 and then advanced distally so that the wire 10 passes laterally outwardly through the lateral port 22 and back into the blood vessel lumen L, as shown in FIG. 3E.

The physician/user of the catheter system of an embodiment can assure that the distal tip of the guide wire 10 and the deflecting port 22 (or other deflecting mechanism) of the deflecting catheter 20 are properly positioned beyond the total occlusion TO without being advanced excessively beyond the end of the total occlusion TO. The proper positioning of the deflecting catheter 20 can vary approximately in the range of 0 cm to 2 cm beyond the distal end of the total occlusion TO, but is not so limited. In one embodiment, for example, the deflecting catheter 20 is positioned approximately 0 to 0.5 cm beyond the end of the total occlusion TO.

As described above, such positioning can in some instances be performed using fluoroscopic imaging. For example, in some instances it may be sufficient to provide suitable radiopaque markers on components of the catheter system that include at least one of the guide wire, the cannula, the deflecting mechanism of the catheter, and some combination of any of these components permitting visual positioning of a distal region of the component via fluoroscopy. Fluorescence imaging is described, for example, in U.S. Pat. Nos. 4,718,417 and 5,106,387.

In addition to fluoroscopy, active imaging systems/methods and other imaging modalities that include, but are not limited to, optical coherence tomography (OCT) and Raman spectroscopy can also be used to provide imaging information in a catheter system. The OCT is described, for example, in U.S. Pat. Nos. 5,321,501, 5,459,570, 5,383,467, and 5,439,000. Raman spectroscopy is described, for example, in International Publication Number WO 92/18008.

The catheter system of an embodiment provides for rotational positioning of the deflecting catheter 20. The rotational positioning allows the direction of deflection of the cannula or guide wire to be selective by allowing the physician/user to aim the deflecting mechanism from the subintimal space back toward the arterial or other blood vessel lumen L.

If the catheter is provided with ultrasonic imaging, such imaging can be used for rotationally positioning the distal tip of the catheter. The catheter of an embodiment is rotationally rigid so that rotation of the proximal end allows for positioning of the distal end. Using the detected presence of the blood vessel lumen, the deflecting port 22, and/or other deflecting mechanisms, the guide wire and/or cannula can be rotationally positioned towards the vessel true lumen via ultrasonically identifiable features of these components.

In an alternative embodiment, a rotationally specific fluoroscopic marker can be provided on the catheter 20, or directly on the cannula. The marker is configured so that the rotational direction of the catheter tip or cannula can be determined by observing the two-dimensional image of the marker using fluoroscopic imaging.

Devices described herein for use in crossing vascular occlusions include catheter systems, also referred to as wire deflection or wire deflecting systems. The wire deflection systems of an embodiment generally comprise a wire deflecting catheter that includes a catheter body and a deflecting cannula. The catheter body includes a proximal end, a distal end, and at least one lumen extending through at least a distal portion of the catheter body. In one embodiment, the lumen communicates with at least one of a distal port and/or a lateral port in at least one of a distal section, distal region, or end zone of the catheter. In various alternative embodiments, the lumen communicates with one or more distal ports and/or one or more lateral ports.

The cannula of an embodiment also includes a proximal end, a distal end, and at least one lumen extending through a distal portion of the cannula. The distal portion of the cannula may include a pre-formed resilient curve, as described below. The cannula is slidably disposed within the lumen of the catheter body but is not so limited. Upon full proximal retraction of the cannula within the catheter body lumen, the distal section of the catheter can be configured to assume at least one of a straight configuration and a curved configuration. The cannula can be deployed through at least one of a lateral port and an end port as appropriate to a procedure to establish a pathway through the subintimal tissue/diffuse disease according to the methods described herein.

The choice of materials and fabrication methods for the catheter shaft and/or the cannula determine the resultant shape of the distal section of the catheter when the cannula is fully retracted. Either the straight or curved configuration of the distal catheter shaft may apply to two embodiments of the catheter as follows. A first embodiment of the catheter includes both a distal and lateral port, and full advancement of the cannula selectively deploys the pre-shaped cannula from the lateral port. A second embodiment of the catheter includes a single distal port, and full advancement of the cannula deploys the pre-shaped cannula from the distal port. Various alternative embodiments include different combinations of lateral ports, distal ports, and cannula deployment options.

The catheter system of an embodiment further comprises a wire configured to pass through the cannula lumen. The wire may be a conventional guide wire, a wire having a sharpened distal tip extended particularly for penetrating the intimal layer of the blood vessel wall and/or diffuse disease of the blood vessel, and/or other wires known in the art. Alternatively, the wire may include passive and/or active visualization or imaging means.

Regarding passive visualization or imaging systems, the catheter body of an embodiment includes one or more fluoroscopically visible markers near the distal end. The markers are configured to permit visual determination of the rotational orientation of the distal end of the catheter body when viewed in a two-dimensional fluoroscopic image. The catheter body can be reinforced to enhance torsional rigidity, and can further comprise a distal nose cone wherein the distal and/or lateral openings may be defined within the nose cone. The distal end of the cannula of an embodiment is pre-formed in a smooth curve which may extend over an arc approximately in the range of 15 to 135 degrees, but is not limited to this range. The pre-formed curve may have a radius approximately in the range of 0.5 millimeters (mm) to 15 mm, but is not limited to this range.

A number of specific embodiments of the catheter system are described below, wherein use of the catheter system is generally described above with reference to FIGS. 3A-3E. These specific embodiments are provided as examples only and do not limit the catheter systems provided herein.

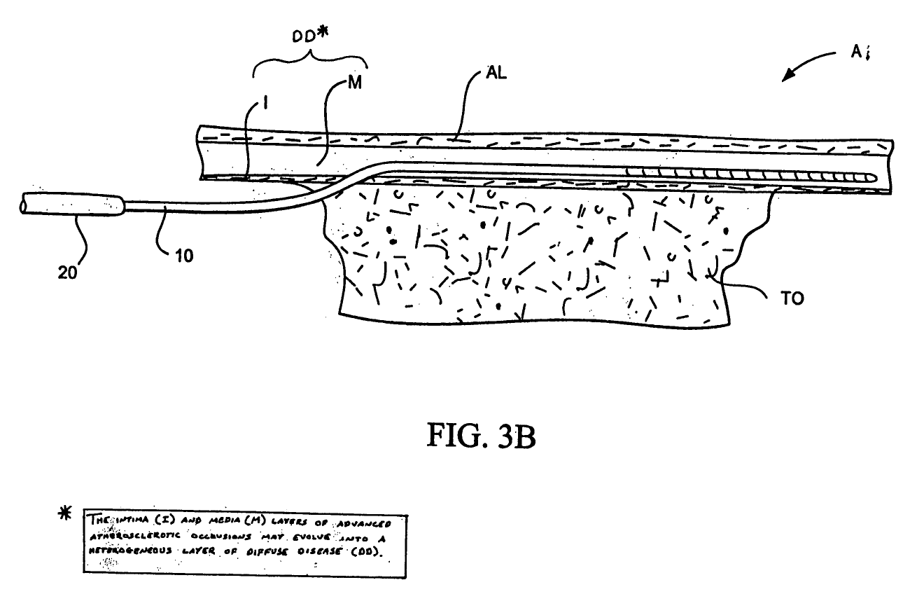

FIG. 4 is a distal region of a deflecting catheter 30, under an embodiment. The deflecting catheter 30 includes a distal end having at least one distal port 32, at least one lateral port 34, and a passive deflecting mechanism 36. The catheter 30 can be advanced over the proximal end of a wire like a guide wire so that the wire passes over the deflecting mechanism 36 and back into the main lumen of the catheter 30. The catheter 30 can then be advanced over the wire until the distal tip enters the subintimal space and approaches the distal end of the wire. By retracting the distal end of the wire within the lumen of catheter. 30 so that its distal tip is proximal to the deflecting mechanism 36, subsequent distal advancement of the wire engages the proximal surface of the deflecting mechanism and causes the wire to be deflected laterally through the lateral port 34. The deflecting catheter 30 of an embodiment is dimensioned as follows, but is not so limited: catheter shaft inner diameter approximately in the range 0.012 inches (to accommodate a 0.010 inch guide wire) to 0.043 inches (to accommodate a 0.039 inch guide wire); and catheter shaft outer diameter approximately in the range 0.020 inches to 0.050 inches.

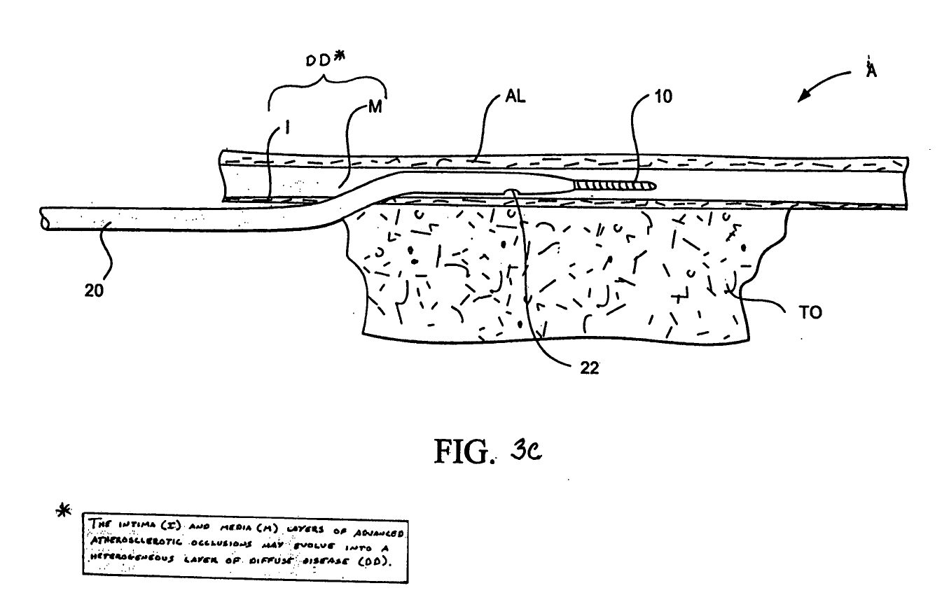

FIG. 5 is a distal region of a deflecting catheter 40, under an alternative embodiment. Deflecting catheter 40 includes at least one distal port 42 and at least one lateral port 44. Instead of a passive deflecting mechanism, deflecting catheter 40 includes an active deflecting mechanism in the form of an axially translatable cannula 46. The cannula 46 is configured to include a resilient pre-formed distal tip that can be advanced through port 44 (shown in broken line). The cannula 46 has a lumen which provides a guide path for the wire. The deflecting catheter 40 of an embodiment is dimensioned as follows, but is not so limited: catheter shaft inner diameter approximately in the range 0.025 inches to 0.055 inches; catheter shaft outer diameter approximately in the range 0.035 inches to 0.065 inches; cannula inner diameter approximately in the range 0.012 inches (to accommodate a 0.010 inch guide wire) to 0.043 inches (to accommodate a 0.039 inch guide wire); and cannula outer diameter approximately in the range 0.020 inches to 0.050 inches.

The cannula 46 of an embodiment is constructed from a composite of braided stainless steel wire that is laminated with polymers such as nylons, urethanes, polyimides or polycarbonates, but is not so limited and can be formed from other materials as appropriate to the medical applications. The distal end of the cannula 46 can be terminated in an angled cut of the material, forming a needle-like tip, or terminated with a sharpened, fluoroscopic hollow metallic tip formed to include Platinum-Iridium, for example. Alternatively, the cannula 46 can be fabricated from a uniform material such as nitinol (nickel-titanium), and terminated in a needle type tip via sharpening into an appropriate shape.

To further increase torque control of the cannula 46, especially in high tortuosity applications, stainless steel wire or other suitable filaments are braided onto the cannula 46 of an embodiment. The braided cannula 46 can also be laminated with appropriate polymers (nylons, polyurethanes) to produce a smooth exterior surface of the cannula shaft. Further, the polymer of an embodiment is coated with a hydrophilic agent to decrease frictional effects as the cannula 46 is translated within the catheter shaft. A resilient curve may be set into the distal section of the cannula 46 via heat setting methods for these materials, such heat setting methods known in the art. Materials and methods of cannula construction also allow some degree of radiopacity, i.e. ability to produce an image under fluoroscopy.

Upon full extension of the cannula 46 from the catheter shaft, the cannula 46 is unconstrained, allowing the as-manufactured curved shape of the distal cannula 46. Upon full retraction of the cannula 46 into the catheter shaft, two configurations are possible. A first configuration is one in which the distal end of the catheter assumes a curved shape. The degree of the curve can range from the as-manufactured curve of the cannula to something approaching a straight configuration. A second configuration is one in which the catheter assumes a straight configuration.

Each of these configurations is possible through the selection of materials used to construct both the distal section of the catheter shaft, and the distal section of the cannula. To achieve the first or curved configuration, the material used to construct the cannula is more robust (for example, nitinol), and the material used to construct the distal section of the catheter shaft is comparitively less robust (for example, a braided shaft laminated with low durometer urethane). This combination allows the catheter shaft to conform more to the shape of the cannula as the cannula is retracted into the catheter shaft. These fabrications and fabrication materials are provided as examples only, as many others allow the distal catheter shaft to follow the shape of the cannula.

The second or straight configuration is achieved by fabricating a less robust or “softer” cannula with wire braid laminated with a medium durometer nylon such as 55D Pebax, and fabricating the catheter shaft with wire braid laminated with polyamide. The softer design of the cannula shaft allows the cannula to conform to the straight configuration of the catheter shaft as the cannula is retracted into the catheter shaft. Alternatively, the distal end of the catheter shaft may be fabricated with an integral section of stainless steel hypotube, or other non-flexible material. This section of hypotube performs in similar fashion to maintain the straight configuration of the distal catheter shaft as the cannula is retracted. Again, these fabrications and fabrication materials are provided as examples only, as many others allow the distal region of the cannula, upon retraction, to conform to the straight configuration of the distal region of the catheter shaft.

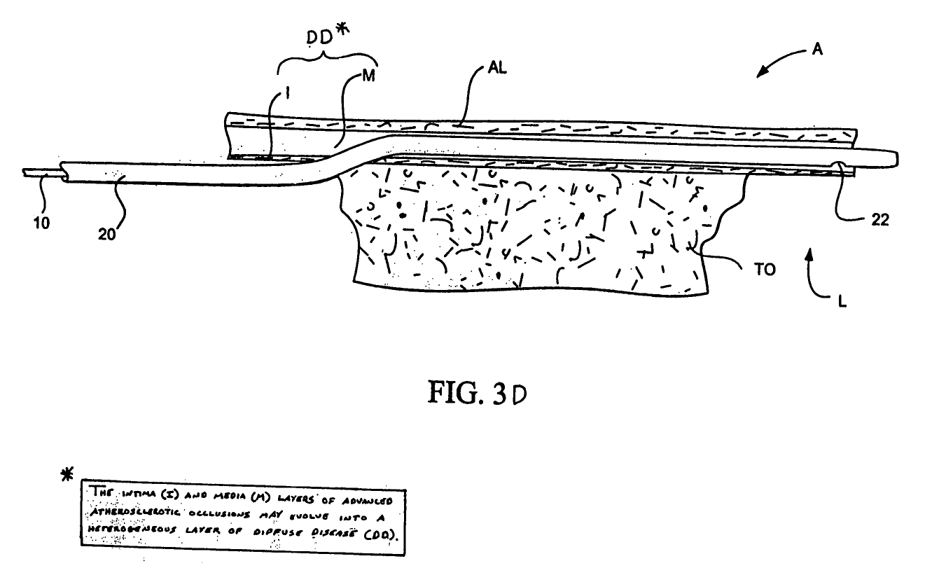

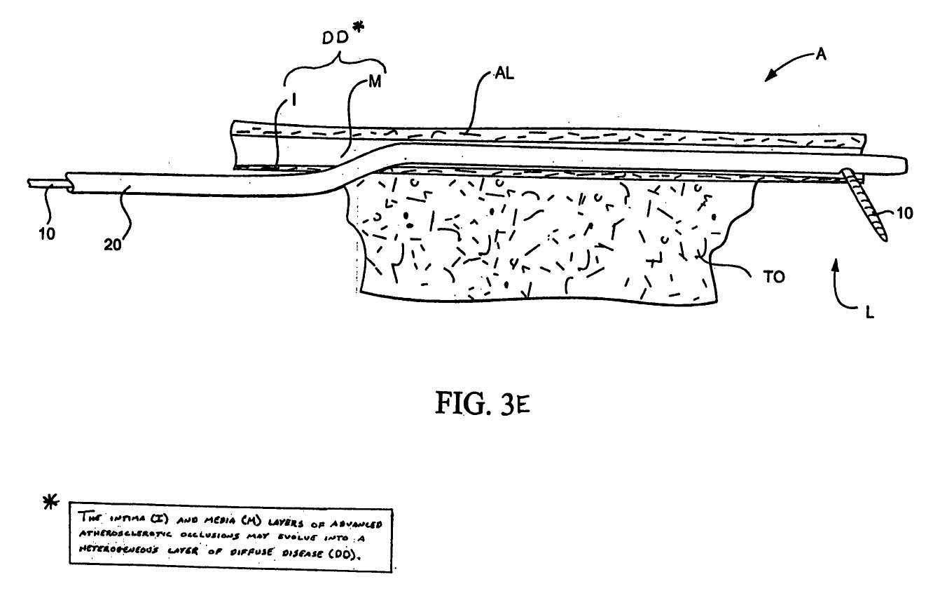

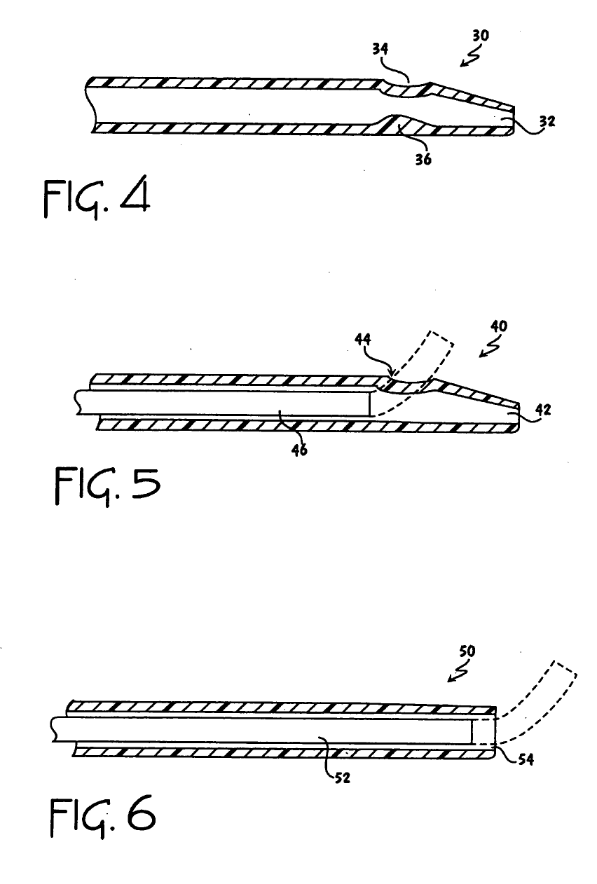

FIG. 6 shows a distal region of a deflecting catheter 50, under another alternative embodiment. The deflecting catheter 50 includes a lumen and at least one distal port 54. A cannula 52 having a pre-formed distal end may be advanced and retracted through the lumen and out of the distal port 54, but the embodiment is not so limited. As described above with reference to the catheter system in FIG. 5, the cannula 52 and catheter shaft of the deflecting catheter 50 can be fabricated of various materials that allow the cannula to assume an as-manufactured curved shape (broken line) when extended from the catheter. The deflecting catheter 50 of an embodiment is dimensioned as follows, but is not so limited: catheter shaft inner diameter approximately in the range 0.025 inches to 0.055 inches; catheter shaft outer diameter approximately in the range 0.035 inches to 0.065 inches; cannula inner diameter approximately in the range 0.012 inches (to accommodate a 0.010 inch guide wire) to 0.043 inches (to accommodate a 0.039 inch guide wire); and cannula outer diameter approximately in the range 0.020 inches to 0.050 inches.

FIG. 6A shows a distal region of a deflecting catheter 50A that assumes a straight configuration in the retracted state, under an alternative embodiment. The materials of the cannula 52A and catheter shaft of the deflecting catheter 50A are fabricated of materials that allow the distal end of the catheter system 50A, when the cannula 52A is fully retracted into the catheter shaft, to assume a straight configuration.

FIG. 6B shows a distal region of a deflecting catheter 50B that assumes a curved configuration in the retracted state, under an alternative embodiment. The materials of the cannula 52B and catheter shaft of the deflecting catheter 50B are fabricated of materials that allow the distal end of the catheter system 50B, when the cannula 52B is fully retracted into the catheter shaft, to assume a curved configuration. Further, the shape of the distal end of the catheter system (either straight 50A or curved 50B, with cannula retracted) determines the type of rotational keying (if any) between the cannula and catheter shaft, and the type of fluoroscopic marking used to assist a physician/user in directing the cannula deployment towards the vessel true lumen.

The three catheter systems 30, 40, and 50 presented with reference to FIGS. 4, 5, and 6 are presented as examples only and do not limit the catheter system to these exact embodiments. A wide variety of other passive and active deflecting mechanisms can be provided on deflecting catheters for use in the methods described herein.

The distal catheter terminations of the catheter system embodiments described herein can be fabricated as a continuation of the catheter shaft polymers, which have been formed or molded into the configurations shown. Alternatively, the distal terminations include a separate nosecone component attached to the terminal end of the catheter shaft. Regarding the attachment of the nosecone, the catheter shaft can be attached to the nosecone by lamination of the shaft polymer onto features at the proximal end of the nosecone, but is not so limited.

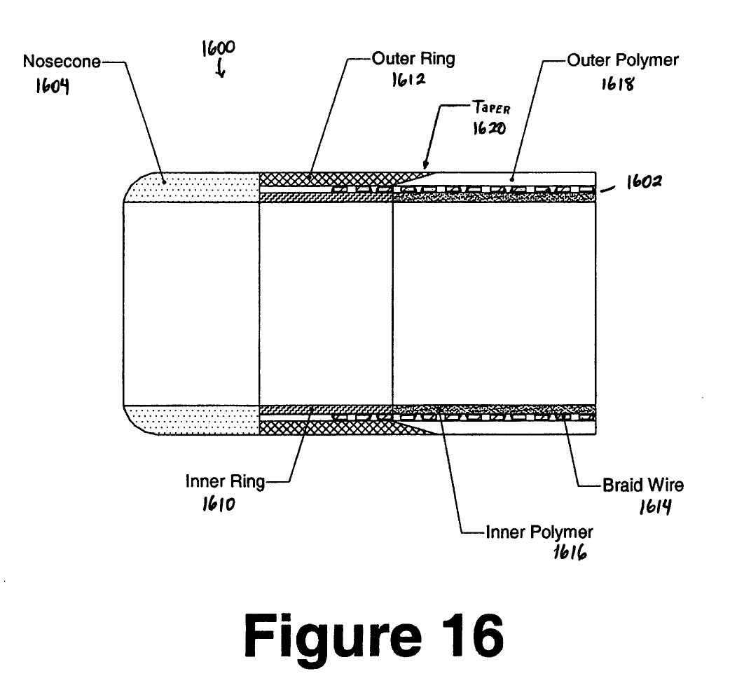

Alternative embodiments of the catheter systems described herein, however, include composite terminations that provide strong flexible connections of the nosecone to the catheter shaft. FIG. 16 shows a composite distal end termination 1600 of a braided catheter shaft 1602 that includes a nosecone 1604, under an embodiment. This composite termination 1600 includes an internal metallic ring 1610 (also referred to as the internal or inner ring 1610) and an external metallic ring 1612 (also referred to as the external or outer ring 1612) between which a braid wire 1614 of the laminated shaft 1602 distally terminates. The internal 1610 and external 1612 rings are each approximately in the range of 1 to 2 mm in length, and have a nominal thickness of approximately 0.002″, but are not so limited. The internal 1610 and external 1612 rings are attached to the braid wire 1614 via at least one of soldering, gluing, and resistance welding, as examples, but other suitable attachment methods can be used. Following attachment of the internal 1610 and external 1612 rings the tubular braid wire 1614 is then laminated with appropriate polymers 1616 and 1618, producing a completed catheter shaft component 1602. This composite termination 1600 of the catheter shaft produces a short, integral metallic “ring”. A nosecone 1604 can be machined with mating features as appropriate and attached to the ring via at least one of welding, gluing and soldering.

The composite termination 1600 provides a very strong, flexible connection of the nosecone 1604 to the catheter shaft 1602. One challenge of terminating the outer polymer layer 1618 to the internal 1610 and external 1612 rings is that if both the shaft polymer 1618 and ring termination are bluntly terminated to each other, the distinct boundary existing betweeri the two materials may tend to delaminate during operation of the catheter system due to bending stresses. The termination of an embodiment alleviates this issue through the inclusion of an internal taper 1620 in the external ring 1612 at the terminal end of the catheter shaft. This taper 1620 of the external ring 1612 allows a small continuous taper of polymer 1618 to be produced underneath the external ring 1612 to afford stress relief upon bending of the catheter shaft 1602 at this boundary location. This taper 1620 prevents delamination of the polymer 1618 from the internal 1610 and external 1612 rings.

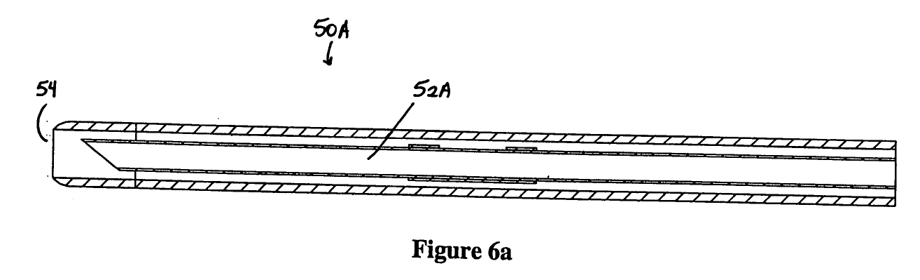

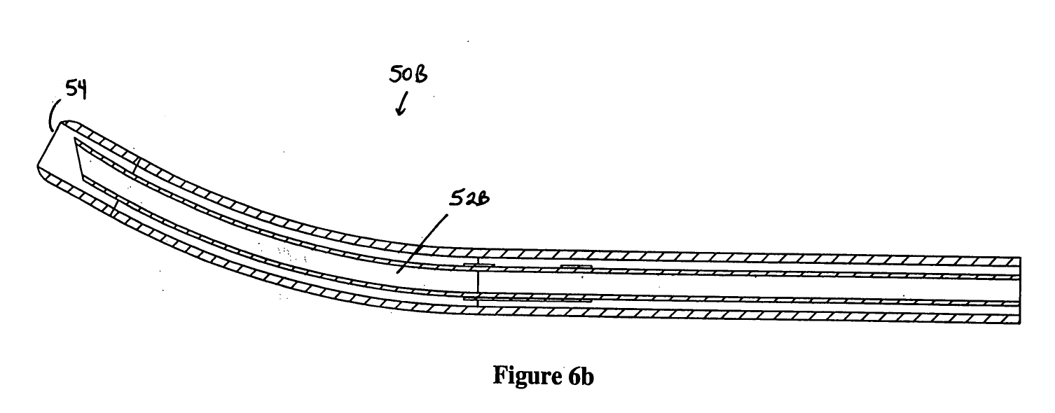

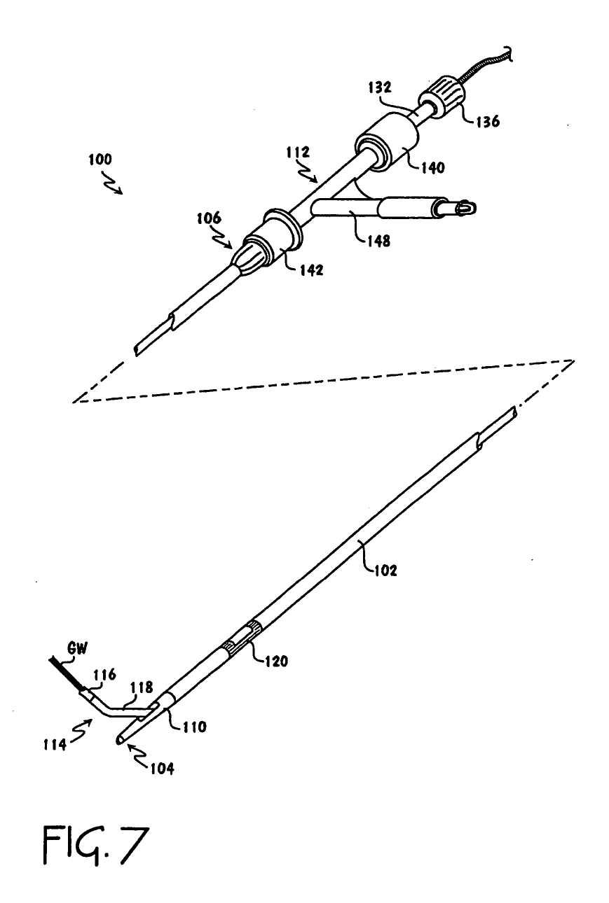

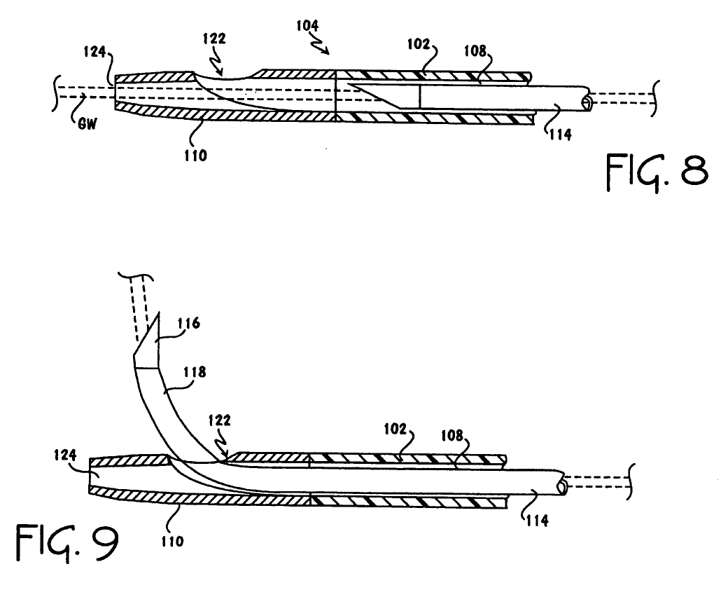

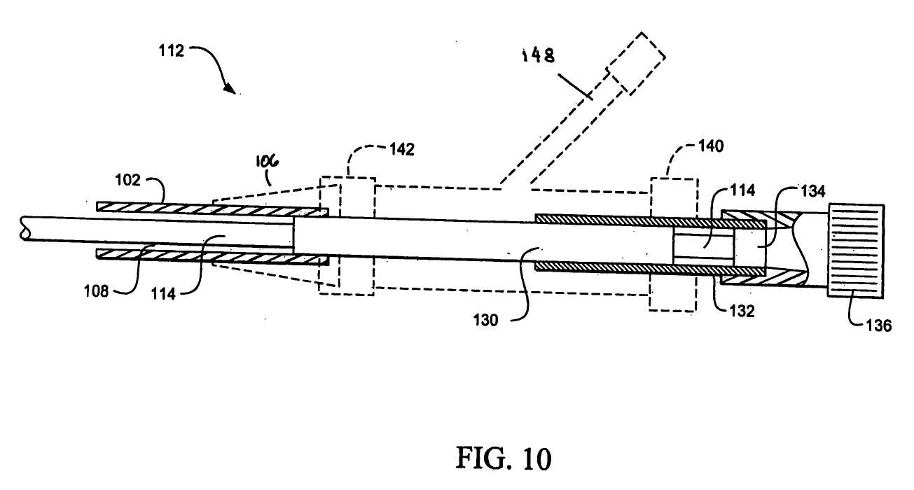

FIG. 7 is a deflecting catheter system 100, under an embodiment. FIG. 8 is a cross-sectional view of a distal region 104 of the defecting catheter system 100 that includes a cannula 114 in a retracted configuration, under an embodiment. FIG. 9 is a cross-sectional view of a distal region 104 of the defecting catheter system 100 that includes a cannula 114 in an advanced configuration, under an embodiment. FIG. 10 is a cross-sectional view of a proximal region 106 of the defecting catheter system 100 that includes a proximal hub 112, under an embodiment.

Referring to FIGS. 7, 8, 9, and 10, the deflecting catheter 100 comprises a catheter body 102 having a distal end 104 and a proximal end 106. Catheter body 102 includes a single lumen 108, and a deflection housing 110 secured to the distal end 104 of the catheter body 102. An actuator hub 112 is secured to the proximal end 106 of the catheter body 102, and an axially translatable cannula 114 is disposed within lumen 108. A distal length 118 of the cannula 114 is pre-formed in a curved shaped, but is not so limited. The cannula 114 has a sharpened tip 116, formed using at least one of metal, hard plastic, composite, and/or combinations of these materials. The cannula tip 116 of an embodiment is radiopaque, but is not so limited. Alternatively or additionally, at lease one separate radiopaque marker, similar to marker 120, is included in the catheter system on the cannula at or near its distal end to facilitate visualization under fluoroscopic imaging. A rotationally specific radiopaque marker 120 is mounted near the distal end of catheter body 102. The marker has a generally U-shaped configuration so that the rotational position of the distal end of the catheter body 102 is apparent when observing the marker using a two-dimensional fluoroscopic image, but the marker is not so limited.

The deflecting catheter 100, in operation, laterally deflects the distal tip of the cannula 114 through a lateral opening 122 in the deflector housing 110. The deflector housing 110 also includes a distal port 124 to permit introduction of the catheter 100 over the proximal end of a guide wire GW, as shown in FIG. 8 in broken line. The guide wire GW passes through the distal port 124 and into the distal end of the cannula 114 and passes through a lumen of cannula 114 to the proximal end of the catheter 100. In one embodiment, the distal length 118 of cannula 114 is straightened and deflected by axially retracting and advancing the cannula 114 between the configurations shown in FIG. 8 and FIG. 9, respectively. Consistent with the description above that references FIG. 5, an alternative embodiment of the catheter system includes a catheter shaft that maintains some degree of curve as the cannula is retracted.

With reference to FIG. 10, the actuator hub 112 comprises a pair of coaxial, telescoping tubes 130 and 132. The outer telescoping tube 132 is connected to a proximal end of cannula 114 using, for example, an adhesive 134. A proximal fitting 136 is further attached to the proximal end of tube 132 so that the assembly of the cannula 114, tube 132, and fitting 136 move together as a unit through the hemostatic fitting 140 at the proximal end of the hub 112. The hub 112 further includes a rotational fitting 142 which permits the catheter body 102 to be rotated relative to the hub body. The cannula 114 and catheter body 102 are rotationally coupled or keyed together to limit and/or prevent relative rotation. The cannula 114 and catheter body 102 of an embodiment are coupled using keying within the hub and/or near the distal end so that rotation of the catheter body 102 causes a like rotation of the cannula 114 as the catheter is rotationally positioned within a blood vessel. A side port 148 is provided on the hub 112 to permit perfiision and/or infusion through the lumen 108 or catheter 102.

FIG. 10A and FIG. 10B are cross-sectional views of a proximal actuation handle 1000 with a locking mechanism that prevents inadvertent deployment of a cannula, under an embodiment. The actuation handle 1000 of an embodiment includes a slide mechanism 1002 with an integral lock 1004 that travels in a linear slot of a handle body 1006. The slide 1002 attaches to the working element 1010 which, in this embodiment, is a cannula 1010. Proximal and distal movement of the slide mechanism advances 1030 and retracts 1040 the working element 1010. Upon full retraction of the slide 1002, the spring-loaded 1008 lock mechanism 1004 is activated, automatically tripping the distal end of the lock 1004 into the distal end of the slot in the handle body 1006 through which the slide translates. Subsequent distal advancement of the slide 1002 and working element 1010 is only possible upon depressing the proximal end of the lock 1004, which disengages the distal end of the lock 1004 from the slot in the handle body 1006. Use of this locking mechanism 1004 prevents the working element 1010 from inadvertent deployment while the catheter system is being tracked within the vasculature. Components like the hemostatic fittings and keying can also be included, as described above with reference to FIG. 10.

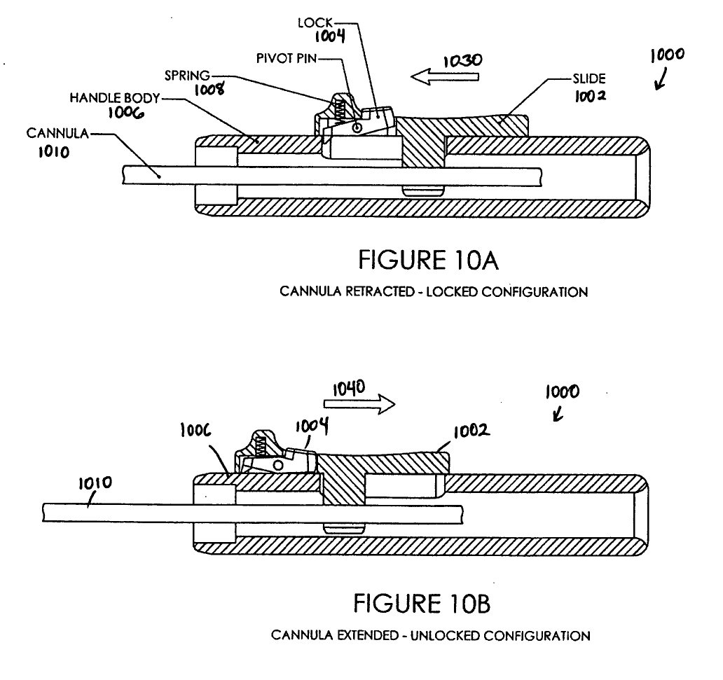

Keying of components of the catheter system, as described above, can be accomplished with a variety of techniques at both the proximal and distal end of the catheter. Keying at the proximal end of the catheter 100 can be achieved in a variety of ways. FIG. 11A and FIG. 11B show rotational keying in cross-sectional views of a proximal region 104 of the defecting catheter system 100, under an embodiment. As an example, telescoping tubes 130 and 132 of the catheter 1102 of an embodiment include asymmetric, mating peripheral geometries having oval cross-sections. Likewise, telescoping tubes 130 and 132 of the catheter 1112 of an alternative embodiment include asymmetric, mating peripheral geometries having triangular cross-sections. These geometries are shown as examples only, and do not limit the catheter systems described herein to these geometries.

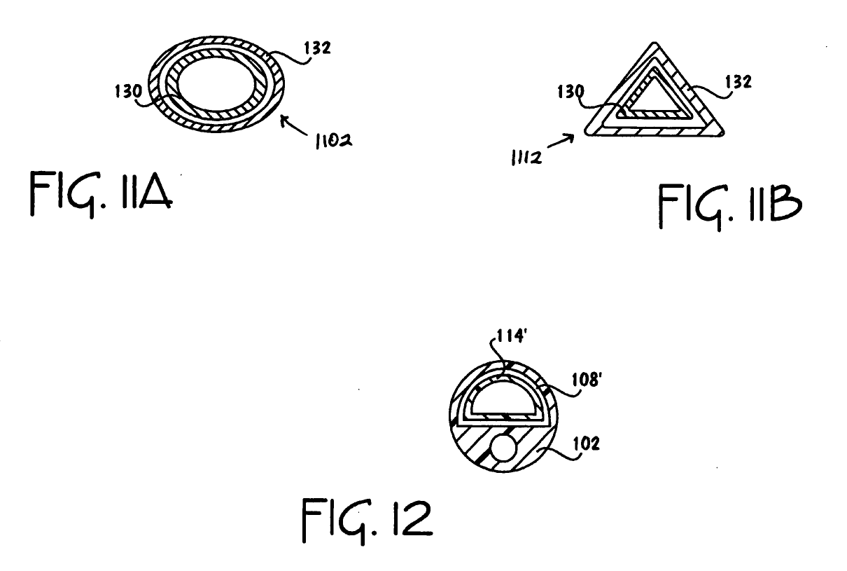

Keying at the distal end of the catheter 100 can also be achieved in a number of ways. FIG. 12 shows rotational keying in cross-sectional views of a distal region of the defecting catheter system 100, under an embodiment. For example, the catheter body 102 can include an asymmetric lumen 108. The cannula 114 uses a mating cross-section, e.g. a D-shaped cross-section in this example. The ability to limit relative rotation of the cannula 114 within the catheter body 102 assures that the curved distal length 118 of the cannula 114 is properly oriented (directed radially outwardly) when the cannula tip 116 emerges through the lateral opening 122.

In use, and with reference to FIG. 8 and FIG. 9, catheter 100 is advanced over guide wire GW while the cannula 114 is retracted. Once the catheter is properly positioned, the guide wire is retracted a few centimeters proximal to the cannula tip 116, and the cannula 114 may be distally advanced. Distal advancement is achieved by forwardly advancing the sleeve 132/hub 136 relative to the body of the hub 112 so that the cannula advances within the lumen 108 of catheter body 102. Prior to advancing the cannula, the lateral port 122 is properly positioned so that it is directed toward the blood vessel lumen. Positioning of the lateral port 122 includes, in one embodiment, rotation of the catheter body 102 using the rotational hub 142. The physician/user observes the marker 120 so that the lateral port 122 is directed in the proper direction, for example radially inward. Following advancement of the cannula into the blood vessel, the guide wire GW can be advanced into the lumen. The cannula 114 is subsequently withdrawn proximally, and the entire catheter assembly is then withdrawn from over the guide wire, leaving the guide wire in place for introduction of other interventional and/or diagnostic catheters.

When the distal end of the catheter system assumes a straight configuration upon retraction of the cannula into the catheter shaft, keying and fluoroscopic marker options are numerous. As described with reference to FIG. 7 above, the distal end of the catheter shaft can specify the direction of cannula deployment via fluoroscopic indicator(s) 120. To support this capability in the catheter system, the cannula and catheter shaft of an embodiment are keyed to each other, as described herein. With the cannula retracted, its “curve” will be straightened, and the fluoroscopic image of the cannula will not indicate the direction of deployment. Therefore, the straightened “curve” rotationally follows (is keyed to) the catheter shaft marker used to indicate the direction the cannula will take when deployed. This rotational keying (alignment of cannula curve to catheter shaft marker 120) is set during the manufacturing of the catheter.

In an alternative embodiment, the features of the fluoroscopic marker 120 are directly incorporated into the catheter nosecone or distal termination of the catheter shaft. Keying of the catheter shaft and cannula is also incorporated as described above.

In another alternative embodiment, the cannula can include a similar marking system 120 as the shaft. Since the cannula marker directly indicates deployment direction of the cannula, keying is not used between catheter shaft and cannula, nor is directional marking on the catheter shaft, but the embodiment is not so limited.

Yet another alternative embodiment includes a combination of catheter shaft marker and cannula marker. In this embodiment, since the cannula includes directional (deployment) marking, the catheter marker is not directional, and it signifies the location of the catheter distal end. A simple fluoroscopic nosecone or ring suffices for this type of marking.

When the distal end of the catheter system assumes a curved configuration upon retraction of the cannula into the catheter shaft, there are also numerous keying and fluoroscopic marker options. In an embodiment, non-directional fluoroscopic markers as described herein are used on either the distal catheter shaft or the distal cannula. Keying is not included since the curve of the cannula in the retracted position automatically angles the distal portion of the catheter shaft in the direction of the cannula deployment.

In an alternative embodiment a directional marker 120 is included on/in the cannula to indicate deployment direction, and works in concert with the directional curve at the distal end of the catheter shaft. Keying is not included in this embodiment (FIG. 6b).

Another alternative embodiment includes a directional marker on the catheter for use in concert with the directional curve at the distal end of the catheter shaft. Keying is included in this embodiment, as described above.

Yet another alternative embodiment includes one or more combinations of the marking schemes described herein. In general, whenever directional marking is used on the catheter shaft, keying is used to align the catheter shaft marker to the cannula curve deployment direction.

In addition to the numerous passive visualization systems described above, various embodiments of the catheter systems described herein can include active on-board visualization systems and methods. One example of an on-board visualization system is described as a rotational ultrasound system in U.S. Pat. Nos. 4,951,677 and 5,000,185, but the on-board visualization systems used in the catheter systems described herein are not so limited.

An embodiment of the catheter system, however, provides ultrasonic or other imaging at or near the total occlusion to assist the physician/user in positioning the catheter system. In one embodiment, guide wire includes at least one ultrasonic imaging component or device to detect the presence and absence of the occluding material as the wire is advanced past the total occlusion. In an alternative embodiment, the deflecting catheter includes such ultrasonic imaging, e.g. in the form of a phased array located near the distal tip of the deflecting catheter. U.S. Pat. Nos. 4,917,097 and 5,368,037 describe a phased array system for use in a deflecting catheter, but the embodiment is not limited to these visualization systems.

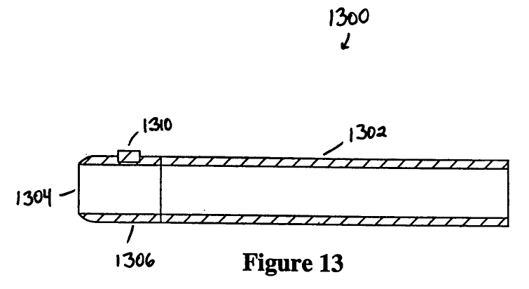

FIG. 13 is a catheter system 1300 that includes a catheter shaft 1302 having a distal end port 1304 and a proximal phased array ultrasound device 1310, under an embodiment. The phased array ultrasound device 1310 is included within and/or on the nosecone 1306 of the catheter system 1300 at the distal end of the catheter shaft 1302, but can be included within/on other components of the catheter system 1300. As the catheter shaft 1302 is rotated, the phased array ultrasound device 1310 produces an image of the surrounding tissue, and the image is used to identify the vessel true lumen. Once the vessel true lumen is correctly identified, a cannula (not shown) is advanced through the lumen 1308 of the catheter shaft 1302; the cannula is keyed to exit in the direction of the vessel true lumen, as identified by the ultrasound image. Alternatively, a rotational imaging catheter system or its functional components are advanced either within the lumen of the catheter system or within the cannula of the catheter system, as appropriate, to a distal site of the catheter from which the surrounding tissue can be imaged.

The visualization system of an embodiment includes ultrasonic imaging guide wires, but is not so limited. For example, U.S. Pat. No. 5,095,911 describes an ultrasonic imaging guide wire, but the embodiment is not limited to this particular imaging-guide wire. As yet another alternative, an imaging guide wire can be advanced to the region of the total occlusion in a direction opposite to that of the guide wire and catheter. In this way the imaging guide wire need not advance through the total occlusion, but could still detect advancement of the catheter and/or guide wire, particularly if ultrasonically opaque components are provided on the catheter and/or the guide wire. In still another alternative, an ultrasonic imaging catheter or guide wire is positioned in a vein adjacent to the arterial occlusion site, allowing imaging of the entire occluded region while the guide wire is advanced through the occluded region.

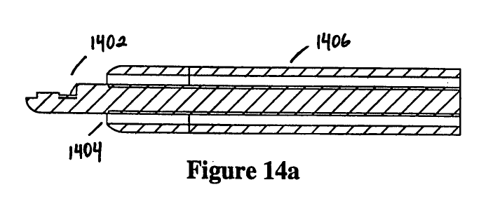

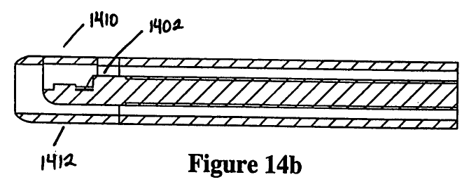

Generally, an ultrasound visualization system can be used under at least two methods. Under a first method, the rotational ultrasound catheter system, or its functional components are advanced first within a catheter lumen to a distal region of the catheter shaft suitable for imaging the surrounding tissue. As an example, the ultrasound components can be advanced beyond a distal end of the catheter. FIG. 14A shows an ultrasound visualization system 1402 deployed beyond a distal end 1404 of the catheter shaft 1406 to image surrounding tissue, under an embodiment.

As another example, the ultrasound components can be to a distal region of the catheter shaft while remaining housed in the catheter. FIG. 14B shows an ultrasound visualization system 1402 deployed to image surrounding tissue from a window 1410 within the nosecone 1412 of the catheter system, under an embodiment. The window can be formed from a polymer like polyethylene that has acoustically transparent properties at the operating frequency of the ultrasound system, but is not so limited.

Following advancement of the ultrasound system to a location appropriate for imaging, the catheter is aligned to the vessel true lumen under a number of methods. Under a first method of catheter alignment, features at the distal end of the catheter, such as those that are machined into a nosecone as the viewing window, are identified via ultrasound imaging, and used to align a lateral or distal port to the true lumen. The ultrasound system is subsequently extracted and a keyed cannula system is advanced within the catheter lumen to exit in the direction of the viewing window and true lumen.

Under a second method of catheter alignment, ultrasound systems/components similar to those described in the first method of catheter alignment are used to align a catheter port with the vessel true lumen. However, instead of relying on a keying mechanism to align the cannula with the vessel true lumen (as described above), use is made of the ultrasonic components of the catheter as fluoroscopic alignment features. In this way, once the catheter port is aligned to the vessel true lumen using the ultrasonic components, the same features may provide fluoroscopic alignment, indicating direction of the vessel true lumen. A cannula with a directional fluoroscopic marker is then advanced within the catheter and brought into fluoroscopic alignment with the catheter marker, providing deployment guidance towards the vessel true lumen.

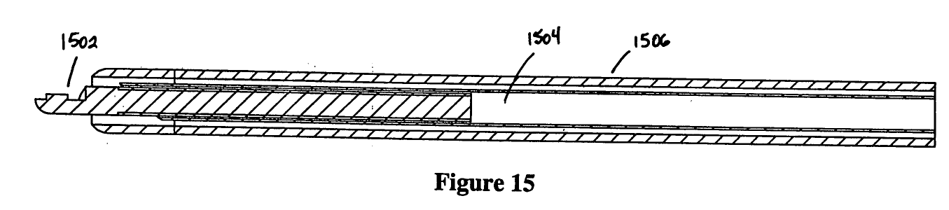

A second method under which ultrasound visualization systems are used includes advancing the rotational ultrasound catheter system or its functional components within the cannula. FIG. 15 shows an ultrasound visualization system 1502 deployed within a cannula 1504 of the catheter shaft 1506 to image surrounding tissue, under an embodiment. Under this method, the ultrasound imaging is used to first identify the vessel true lumen via either images taken with the ultrasound system extended out from the distal port or ultrasound images taken through a lateral window at the distal end of the catheter shaft. Further, the ultrasound imaging system may be retracted slightly to identify features on the cannula shaft or tip which indicate cannula deployment direction. The cannula can then be rotated to bring the identified cannula features into alignment with the vessel true lumen, as appropriate. The cannula is then deployed to gain access to the vessel true lumen.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the terms “herein,” “hereunder,” “above,” “below,” and terms of similar import, when used in this application, refer to this application as a whole and not to any particular portion of this application. When the word “or” is used in reference to a list of two or more items, that word covers all of the following interpretations of the word: any of the items in the list, all of the items in the list and any combination of the items in the list.

The above description of illustrated embodiments of the catheter system is not intended to be exhaustive or to limit the catheter system to the precise form disclosed. While specific embodiments of, and examples for, the catheter system are described herein for illustrative purposes, various equivalent modifications are possible within the scope of the catheter system, as those skilled in the relevant art will recognize. The teachings of the catheter system provided herein can be applied to other medical devices and systems, not only for the catheter systems described above.

The elements and acts of the various embodiments described above can be combined to provide further embodiments of the catheter system. These and other changes can be made to the catheter system in view of the above detailed description.

All of the above references and United States patents and patent applications are incorporated herein by reference. Aspects of the catheter system can be modified, if necessary, to employ the systems, functions and concepts of the various patents and applications described above to provide yet further embodiments of the system.

In general, in the following claims, the terms used should not be construed to limit the catheter system to the specific embodiments disclosed in the specification and the claims, but should be construed to include all catheter systems and medical devices that operate under the claims to cross vascular occlusions. Accordingly, the catheter system is not limited by the disclosure, but instead the scope of the catheter system is to be determined entirely by the claims.

While certain aspects of the catheter system are presented below in certain claim forms, the inventors contemplate the various aspects of the catheter system in any number of claim forms. Accordingly, the inventors reserve the right to add additional claims after filing the application to pursue such additional claim forms for other aspects of the catheter system.

1. A catheter system for use in forming a pathway between an extraluminal space within a blood vessel and a true lumen of the blood vessel, comprising:
a catheter body including at least one lumen configured to track over a guide wire to a treatment site;
a catheter endpiece coupled to the distal end of the catheter body, the catheter endpiece including at least one port in communication with the lumen; and
a working element having a distal end configured to deploy through the port for delivery from a first vascular location within the extraluminal space to a second vascular location within the true lumen of the blood vessel when the working element is advanced distally through the port, wherein a distal end of the catheter body assumes one of a first and a second configuration when the working element is proximally retracted into the catheter body.

2. The catheter system of claim 1, wherein the first configuration is a straight configuration.

3. The catheter system of claim 1, wherein the second configuration is a curved configuration.

4. The catheter system of claim 1, wherein an inside diameter of the lumen is uniform.

5. The catheter system of claim 1, wherein the working element includes a cannula having at least one lumen.

6. The catheter system of claim 5, wherein the cannula includes braided tubular composition.

7. The catheter system of claim 1, wherein the working element includes at least one guide wire.

8. The catheter system of claim 1, wherein the working element includes a cannula and at least one guide wire, wherein the guide wire is slidably disposed within the cannula.

9. The catheter system of claim 1, wherein the working element includes a sharpened distal tip.

10. The catheter system of claim 1, wherein the port is positioned at a distal end of the catheter endpiece.

11. The catheter system of claim 1, wherein the port is positioned proximal to a distal end of the catheter endpiece.

12. The catheter system of claim 1, wherein the at least one port includes a first port positioned at a distal end of the catheter endpiece, and a second port positioned in at least one region proximal to the distal end of the catheter endpiece.

13. The catheter system of claim 1, wherein the distal end of the working element includes a preformed resilient tip.

14. The catheter system of claim 1, further comprising at least one guide wire.

15. The catheter system of claim 1, further comprising at least one component of an active visualization system.

16. The catheter system of claim 1, further comprising at least one component of a passive visualization system.

17. The catheter system of claim 16, wherein the component includes at least one component of a fluoroscopic marking system positioned on the catheter body.

18. The catheter system of claim 16, wherein the component includes at least one component of a fluoroscopic marking system positioned on the working element.

19. The catheter system of claim 1, further comprising a deflection mechanism in communication with the lumen and the port.

20. The catheter system of claim 1, wherein the extraluminal space is located within diffuse disease of the blood vessel.

21. The catheter system of claim 1, wherein the extraluminal space is located between an adventitial layer and an intimal layer of the blood vessel.

22. The catheter system of claim 1, wherein the catheter body includes a tubular wire braid laminated with polymer.

23. The catheter system of claim 22, wherein the catheter shaft is terminated with at least one internal ring and at least one external ring, wherein the internal ring is connected to an internal surface of the tubular wire braid and the external ring is connected to an external surface of the tubular wire braid.

24. The catheter system of claim 23, wherein a proximal internal surface of the external ring is internally tapered so that polymer of the catheter body continues into the taper.

25. The catheter system of claim 1, wherein the working element is connected to a linear slide mechanism in a proximal handle.

26. The catheter system of claim 25, wherein the slide mechanism locks when fully retracted and prevents inadvertent actuation of the working element.

27. A re-entry catheter for use in forming a pathway between an extraluminal space within a blood vessel and a true lumen of the blood vessel, comprising:
a catheter body including at least one lumen configured to track over a guide wire to a treatment site;
a catheter endpiece coupled to the distal end of the catheter body, the catheter endpiece including at least one port in communication with the lumen, and
a working element having a distal end configured to deploy through the port for delivery from a first vascular location within the extraluminal space to a second vascular location within the true lumen of the blood vessel when the working element is advanced distally through the port, wherein a distal end of the catheter body assumes a curved configuration when the working element is proximally retracted into the catheter body.

28. A re-entry catheter for use in forming a pathway between an extraluminal space within a blood vessel and a true lumen of the blood vessel, comprising:
a catheter body including at least one lumen configured to track over a guide wire to a treatment site;
a catheter endpiece coupled to the distal end of the catheter body, the catheter endpiece including at least one port in communication with the lumen, and
a working element having a distal end configured to deploy through the port for delivery from a first vascular location within the extraluminal space to a second vascular location within the true lumen of the blood vessel when the working element is advanced distally through the port, wherein a distal end of the catheter body assumes a straight configuration when the working element is proximally retracted into the catheter body.

29. A catheter system for use in forming a pathway between an extraluminal space within a blood vessel and a lumen of the blood vessel, comprising:
a catheter body configured for advancement over a wire into the extraluminal space to a re-entry target location of the blood vessel lumen, the catheter body including at least one distal port and at least one lumen;
a cannula selectively disposed within the at least one lumen and configured for slidable deployment from the distal port of the catheter for use in generating a passage from the extraluminal space into the blood vessel lumen;
an active visualization device configured for deployment to a distal region of the catheter system via at least one of the lumen and the cannula for generating images for use in positioning at least one of the cannula and the distal port of the catheter body relative to the re-entry target location; and
a guide wire positioned in at least one of the catheter body lumen and the cannula and configured for advancement into the blood vessel lumen via the passage.

30. The catheter system of claim 29, wherein a distal end of the catheter body assumes one of a first and a second configuration when the cannula is proximally retracted into the catheter body.

31. The catheter system of claim 30, wherein the first configuration is a straight configuration.

32. The catheter system of claim 30, wherein the second configuration is a curved configuration.

33. The catheter system of claim 29, wherein an inside diameter of the lumen is uniform.

34. The catheter system of claim 29, wherein the cannula includes a sharpened distal tip.

35. The catheter system of claim 29, wherein the distal port is positioned at a distal end of the catheter body.

36. The catheter system of claim 29, wherein the distal port is positioned proximal to a distal end of the catheter body.

37. The catheter system of claim 29, wherein the at least one distal port includes a first port positioned at a distal end of the catheter body, and a second port positioned in at least one region proximal to the distal end of the catheter body.

38. The catheter system of claim 29, wherein a distal end of the cannula includes a preformed resilient tip.

39. The catheter system of claim 29, further comprising at least one component of a passive visualization system.

40. The catheter system of claim 39, wherein the component of a passive visualization system includes at least one component of a fluoroscopic marking system positioned on the catheter body.

41. The catheter system of claim 39, wherein the component of a passive visualization system includes at least one component of a fluoroscopic marking system positioned on the cannula.

42. The catheter system of claim 29, further comprising a deflection mechanism in communication with the catheter body lumen and the distal port.

43. The catheter system of claim 29, wherein the extraluminal space is located within diffuse disease of the blood vessel.

44. The catheter system of claim 29, wherein the extraluminal space is located between an adventitial layer and an intimal layer of the blood vessel.

45. The catheter system of claim 29, wherein the images include images of vascular tissue surrounding a distal end of the catheter body.

46. The catheter system of claim 29, wherein the active visualization device includes at least one of a rotational imaging device, an ultrasonic imaging device, an optical coherence tomography device, and a phased array imaging device.

47. The catheter system of claim 29, wherein the active visualization device is configured for delivery via a lumen of the cannula to a distal end of the catheter body.

48. The catheter system of claim 29, wherein the active visualization device is configured for delivery via a lumen of the catheter to a distal end of the catheter body.

49. The catheter system of claim 29, wherein the cannula includes at least one feature that is identifiable using the active visualization device and is in registration with a deployment direction of the cannula from the distal port.

50. The catheter system of claim 29, wherein a distal region of the catheter body includes at least one feature that is identifiable using the active visualization device and is in registration with a deployment direction of the cannula from the distal port.

51. The catheter system of claim 29, wherein the catheter body and the cannula are keyed together.

52. A catheter system for use in forming a pathway between an extraluminal space within a blood vessel and a lumen of the blood vessel, comprising:
a catheter body configured for advancement over a wire into the extraluminal space to a re-entry target location of the blood vessel lumen, the catheter body including at least one distal port and at least one lumen;
a cannula selectively disposed within the at least one lumen and configured for slidable deployment from the distal port of the catheter for use in generating a passage from the extraluminal space into the blood vessel lumen;
an active visualization device on at least one distal end region of the catheter body, wherein the active visualization device generates images for use in positioning at least one of the cannula and the distal port of the catheter body relative to the re-entry target location; and
a guide wire positioned in at least one of the catheter body lumen and the cannula and configured for advancement into the blood vessel lumen via the passage.

53. A catheter system for use in forming a pathway between an extraluminal space within a blood vessel and a lumen of the blood vessel, comprising:
means for forming a track from the blood vessel lumen into an extraluminal space between an intimal layer and an adventitial layer of the blood vessel, wherein the track extends from proximal of an occlusion in the blood vessel to a point distal to the occlusion; and
means for selectively forming a passage from the track back into the blood vessel lumen at a re-entry location distal to the occlusion via at least one port of a catheter system.

54. The catheter system of claim 53, further comprising means for identifying the re-entry location.

55. The catheter system of claim 53, wherein the means for selectively forming a passage further includes means for selectively deploying a working element from a distal region of the catheter system.

56. The catheter system of claim 53, wherein the means for selectively forming a passage further includes means for selectively aligning a working element of the catheter system with the re-entry location.

57. The catheter system of claim 53, further comprising means for tracking the catheter system to a site proximate to the occlusion.

58. A catheter system for use in forming a passage between an extraluminal space within tissue of a blood vessel and a true lumen of the blood vessel, comprising:
means for advancing the catheter system into the extraluminal space at a location proximal to an occlusion in the blood vessel;
means for positioning a working element in the catheter system;
means for identifying a location for re-entering the blood vessel lumen from the extraluminal space, wherein the re-entry location is distal to the occlusion;
means for positioning at least one port of the catheter system towards the re-entry location; and
means for selectively forming a passage from the extraluminal space back into the blood vessel lumen at the re-entry location via the port.
